(19) United States
(12) Patent Application Publication
(10) Pub. No.: US 2026/0325464 A1
(43) Pub. Date: Oct. 22, 2026

(54) COMPOSITIONS FOR MODULATING THE EXPRESSION OF AQUAPORINS IN NEURODEGENERATIVE DISORDERS

(71) Applicants: ANJU MAJEED, Bangalore (IN); SHAHEEN MAJEED, Elk Ridge, UT (US); ANJALI PANDEY, Bangalore (IN); SARANG BANI, Bangalore (IN)

(72) Inventors: ANJU MAJEED, Bangalore (IN); SHAHEEN MAJEED, Elk Ridge, UT (US); ANJALI PANDEY, Bangalore (IN); SARANG BANI, Bangalore (IN)

(21) Appl. No.: 19/478,826

(22) PCT Filed: Apr. 29, 2024
(86) PCT No.: PCT/US24/26820

§ 371 Date: Oct. 26, 2025

(30) Foreign Application Priority Data
Apr. 29, 2023 (IN) 202341030814

(51) Int. Cl. A61K 31/12; A61P 25/16; A61P 25/28

(52) U.S. Cl. CPC A61K 31/12; A61P 25/16; A61P 25/28

(57) ABSTRACT

The present invention discloses a composition comprising compounds of Formula I for use in modulating and maintaining water homeostasis in the brain by modulating the expression of aquaporins. More specifically, the invention discloses the use of Gingerenone A in the management of neurodegenerative disorders, specifically Alzheimer's disease and Parkinson's disease by modulating aquaporin expression.

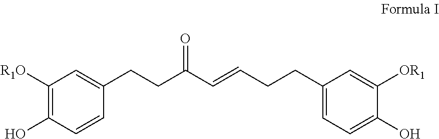

### CROSS REFERENCE TO RELATED APPLICATIONS

This application is a US national stage application of PCT application no. PCT/US24/26820 filed on 29 Apr. 2024, claiming priority from Indian Provisional application no. IN202341030814, filed on 29 Apr. 2023, the contents of which is incorporated herein.

### FIELD OF INVENTION

The present invention in general relates to compositions comprising compounds of Formula I More specifically, the invention relates to compositions comprising Gingerenone A. Further specifically, the invention relates to a compositions comprising Gingerenone A and methods thereof for use in modulating the expression of Aquaporins (AQP) in neurodegenerative disorders, specifically Alzheimer's disease and Parkinson's disease.

### BACKGROUND OF THE INVENTION

Water is the single largest constituent in the body weighing about 55-66% of the total body weight, distributed between the intracellular and extracellular components. The solutes in the water determine the osmotic pressure of the cell, which should be equal between intracellular and extracellular fluids. Thus, water should be transported easily between plasma membranes to maintain the osmotic pressure of the cells. Imbalance in the concentration of either the water or the solutes increase osmotic stress (hyperosmolarity and hypoosmolality) which is the primary cause for the developments of various diseases and disorders. Hyperosmolarity triggers cell shrinkage, oxidative stress, protein carbonylation, mitochondrial depolarization, DNA damage, and cell cycle arrest, thus rendering cells susceptible to apoptosis. Hypoosmotic stress can act as an inflammatory stimulus and is also associated with a number of disorders, including acetaminophen toxicity and brain edema (Brocker et al., The role of hyperosmotic stress in inflammation and disease, Biomol Concepts. 2012 August; 3(4): 345-364). Thus, maintaining a stable water homeostasis is important to sustain a constant osmotic balance of the cells for maintaining general health and wellbeing.

Aquaporins (AQPs) are part of the family of the integral membrane proteins with a function to transport water, glycerol, ammonia, urea, H₂O₂, and other small molecules across the biological membranes. The expression levels of AQPs are essential for maintaining water homeostasis and they have a relevant role in the development of many diseases. Recent discoveries suggest, that AQPs play an important role in the process of fat accumulation, regulation of oxidative stress, and two crucial aspects of insulin resistance, type-2 diabetes and obesity. Many AQPs viz AQP1, AQP4, AQP9, AQP17 have been implicated in diseases like diabetes, insulin resistance, neurodegenerative diseases like Alzheimer's disease, Parkinson's disease, loss of vision, loss of skin barrier function, kidney diseases, xerostomia (dry mouth), and the edema that follows stroke or trauma to the brain or spinal cord etc (Salman et al., Recent breakthroughs and future directions in drugging aquaporins, Trends in Pharmacological Sciences, Volume 43, Issue 1, January 2022, Pages 30-42). Modulating the expression of these aquaporins has been touted as a possible drug target for the management of these diseases.

Several small molecules like 3,3′-(1,3-Phenylene)bis(2-propenoic acid), N-1,3,4-Thiadiazol-2-yl-3-pyridinecarboxamide, were studied for modulating the activity and expression of AQPs (Salman et al., Recent breakthroughs and future directions in drugging aquaporins, Trends in Pharmacological Sciences, Volume 43, Issue 1, January 2022, Pages 30-42). Natural molecules like Bacopaside from Bacopa monneri has also been reported to modulate the expression of aquaporins. However, most of the therapeutic interventions rely on managing the symptoms and currently no pharmacological interventions exist for any pathologies of aquaporin dysfunction. There exists an unmet clinical need for identifying new drug development frameworks and small molecules for conditions associated with imbalances in water and solute homeostasis and modulating aquaporin expression. The present invention solves the unmet need by disclosing a natural plant based compounds for modulating the expression of different aquaporins and in the management of aquaporin related diseases like neurodegenerative diseases, metabolic syndrome, cardiovascular diseases, cancer, inflammatory diseases, allergy, diabetes, insulin resistance, renal disorders, skin hydration, brain edema, immune system disorders, obesity, and liver steatosis, more specifically, for the management of neurodegenerative diseases like Alzheimer's disease, Parkinson's disease, Vascular dementia, neuromyelitis optica, Amyotrophic lateral sclerosis, prion diseases, idiopathic-normal pressure hydrocephalus, Lewy body dementia and fronto-temporal dementia.

It is the principle object of the invention to disclose a composition comprising compounds of Formula I, wherein R₁ is methyl.

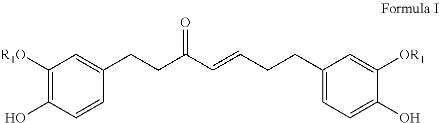

It is another object of the invention to disclose the use of a composition comprising compounds of Formula I and methods thereof for modulating the expression of aquaporins in a subject.

It is another object of the invention to disclose the use of a composition comprising compounds of Formula I and methods thereof for increasing and maintaining water homeostasis in the brain of a subject with neurodegenerative disease.

It is yet another object of the invention to disclose the use of a composition comprising compounds of Formula I and methods thereof for management of Alzheimer's disease in a subject.

It is yet another object of the invention to disclose the use of a composition comprising compounds of Formula I and methods thereof for management of Parkinson's disease in a subject.

The present invention solves the above mentioned objects and provides further related advantages.

### SUMMARY OF THE INVENTION

In a most preferred embodiment, the invention discloses a composition comprising compounds of Formula I.

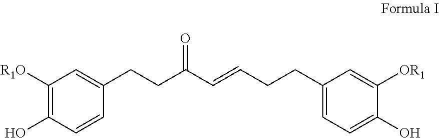

In a related aspect, the compound of Formula I is Gingerenone A, when R₁ is methyl in Formula I.

In another most preferred embodiment, the invention discloses a composition comprising compound of Formula I and methods thereof for modulating the expression of aquaporins in a subject. In a related aspect, the compound is Gingerenone A, when R₁ is methyl in Formula I

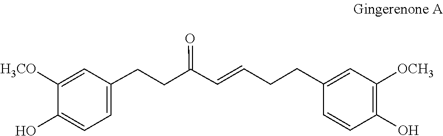 [SMILES: COc1cc(CC/C=C/C(=O)CCc2ccc(O)c(OC)c2)ccc1O]

In another most preferred embodiment, the invention discloses a composition comprising compounds of Formula I and methods thereof for increasing water homeostasis in the brain of a subject with neurodegenerative conditions. In a related aspect, the compound is Gingerenone A, when R1 is methyl in Formula I.

In yet another most preferred embodiment, the invention discloses a composition comprising compounds of Formula I and methods thereof in therapeutic management of Alzheimer's disease in a subject. In a related aspect, the compound is Gingerenone A, when R₁ is methyl in Formula I.

In yet another most preferred embodiment, the invention discloses a composition comprising compounds of Formula I and methods thereof for therapeutic management of Parkinson's disease in a subject. In the related aspect, the compound is Gingerenone A, when R₁ is methyl in Formula I.

### BRIEF DESCRIPTION OF DRAWINGS

FIG. 1 is a graphical representation of modulation of aquaporin 4 expression in brain cell supernatants of experimental animals with Alzheimer's pathology and administered with Gingerenone A (5, 10 and 20 mg/kg)

FIG. 2 is a graphical representation of modulation of tau levels in plasma of experimental animals with Alzheimer's pathology and administered with Gingerenone A (5, 10 and 20 mg/kg)

FIG. 3 is a histopathological section of brain of animals (vehicular controls) in AD experiments showing frontal cortex (FIG. 3A) and hippocampal sections of CA1 (FIG. 3B), CA2 (FIG. 3C) and CA3 (FIG. 3D) regions.

FIG. 4 is a histopathological section of brain of animals (Alzheimer's disease (AD) controls) in AD experiments showing frontal cortex (FIG. 4A) and hippocampal sections of CA1 (FIG. 4B), CA2 (FIG. 4C) and CA3 (FIG. 4D) regions.

FIG. 5 is a histopathological section of brain of animals in AD experiments administered with Gingerenone A (5 mg/kg body weight) showing frontal cortex (FIG. 5A) and hippocampal sections of CA1 (FIG. 5B), CA2 (FIG. 5C) and CA3 (FIG. 5D) regions.

FIG. 6 is a histopathological section of brain of animals in AD experiments administered with Gingerenone A (10 mg/kg body weight) showing frontal cortex (FIG. 6A) and hippocampal sections of CA1 (FIG. 6B), CA2 (FIG. 6C) and CA3 (FIG. 6D) regions.

FIG. 7 is a histopathological section of brain of animals in AD experiments administered with Gingerenone A (20 mg/kg body weight) showing frontal cortex (FIG. 7A) and hippocampal sections of CA1 (FIG. 7B), CA2 (FIG. 7C) and CA3 (FIG. 7D) regions.

FIG. 8 is a histopathological section of brain of animals in AD experiments administered with Donepezil showing frontal cortex (FIG. 8A) and hippocampal sections of CA1 (FIG. 8B), CA2 (FIG. 8C) and CA3 (FIG. 8D) regions.

FIG. 9 is a graphical representation of modulation of aquaporin 4 expression in brain cell supernatants of experimental animals with Parkinsons's pathology and administered with Gingerenone A (5, 10 and 20 mg/kg).

FIG. 10 is a histopathological section of brain of animals (vehicular controls) in PKD experiments showing substantia niagara (FIG. 10A, FIG. 10B).

FIG. 11 is a histopathological section of brain of animals (PKD controls) in PKD experiments showing substantia niagara (FIG. 11A, FIG. 11B).

FIG. 12 is a histopathological section of brain of animals in PKD experiments administered with Gingerenone A (5 mg/kg body weight) showing substantia niagara (FIG. 12A, FIG. 12B)

FIG. 13 is a histopathological section of brain of animals in PKD experiments administered with Gingerenone A (10 mg/kg body weight) showing substantia niagara (FIG. 13A, FIG. 13B)

FIG. 14 is a histopathological section of brain of animals in PKD experiments administered with Gingerenone A (20 mg/kg body weight) showing substantia niagara (FIG. 14A, FIG. 14B)

FIG. 15 is a histopathological section of brain of animals in PKD experiments administered with carbidopa showing substantia niagara (FIG. 15A, FIG. 15B)

### DETAILED DESCRIPTION OF THE PREFERRED EMBODIMENTS

### Selected Definitions

All the terms used in this application carry ordinary meaning as known in the prior art unless otherwise specified. Few other specific definitions used in this invention are explained below, which applies throughout this specification. Claims provide broader definition unless and otherwise specified.

Furthermore, the terms “approximately,” “approximate,” “about,” and similar terms generally refer to ranges that include the identified value within a margin of 20%, 10%, or preferably 5%, and any values there between.

As used herein, treatment or management of a condition refers to effectively ameliorating conditions disclosed in the invention. An effective dose refers to positive or modulatory effects of a condition in a subject covered under this invention.

As used herein, PKD refers to Parkinson's Disease, AD refers to Alzheimer's Disease (AD), and CA refers to Cornu Ammonis.

As used herein, a composition comprising compounds of Formula I

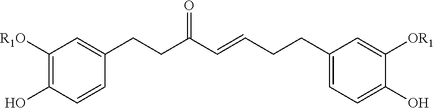

Wherein R₁ is methyl or H. In all the embodiments of the invention, without any limitations, it includes structures of both the compounds of Formula I.

In a most preferred embodiment, the invention discloses a composition comprising compounds of Formula I.

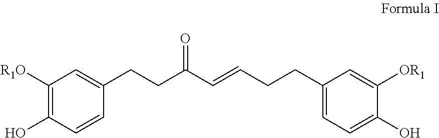

In a related aspect, the compound is Gingerenone A, when R₁ is methyl in Formula I.

In a preferred embodiment of the invention, Gingerenone A is isolated from plant sources or chemically synthesised. In a related embodiment of the invention, the plant source is preferably, but not limited to, Zingiber officinale, Alnus hirsuta, Alnus sibirica, Alnus glutinosa, Alnus formosana, Alnus japonica, Alnus nepalensis, Viscum cruciatum. In a related embodiment of the invention, wherein the composition further comprises carriers and excipients. In a related embodiment of the invention, the composition is formulated with a pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a method for modulating the expression of aquaporins in a subject with a disease condition comprising a) identifying the subject with the disease condition, b) administering an effective dose of a composition comprising compounds of Formula Ito the said subject to bring about a change in the expression of aquaporins. In a related embodiment of the invention, the compound is Gingerenone A. In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In a related embodiment of the invention, the aquaporins are selected from the group consisting of AQP4 and AQP9. In a related embodiment of the invention, modulating the expression of aquaporins improves water homeostasis in the subject. In another related embodiment of the invention, the diseases condition is selected from the group consisting of neurodegenerative diseases, metabolic syndrome, cardiovascular diseases, cancer, inflammatory diseases, allergy, diabetes, insulin resistance, renal disorders, skin hydration, brain edema, immune system disorders, obesity, and liver steatosis. In a preferred embodiment of the invention, modulating the expression of aquaporins is useful for management of diseases and disorders selected from the group consisting of neurodegenerative diseases, diabetes, and insulin resistance. In another related embodiment of the invention, the effective dose of the compounds is in the range 5-30 mg/kg bodyweight of the subject. In another preferred embodiment of the invention, the subject is a mammal. In a related embodiment of the invention, the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a composition comprising compounds of Formula I for use in modulating the expression of aquaporins in a subject with a disease condition. In a related embodiment of the invention, the compound is Gingerenone A, when R₁ is methyl in Formula I. In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In a related aspect, the aquaporins are selected from the group consisting of AQP4 and AQP9. In a related embodiment of the invention, modulating the expression of aquaporins improves water homeostasis in the subject. In another related embodiment of the invention, the disease condition is selected from the group consisting of neurodegenerative diseases, metabolic syndrome, cardiovascular diseases, cancer, inflammatory diseases, allergy, diabetes, insulin resistance, renal disorders, skin hydration, brain edema, immune system disorders, obesity, and liver steatosis. In a preferred embodiment of the invention, modulating the expression of aquaporins is useful for management of diseases and disorders selected from the group consisting of neurodegenerative diseases, diabetes, and insulin resistance. In another related embodiment of the invention, the effective dose of the compounds is in the range 5-30 mg/kg bodyweight of the subject. In another preferred embodiment of the invention, the subject is a mammal. In a related embodiment of the invention, the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses use of a composition comprising compounds of Formula I for modulating the expression of aquaporins in a subject with a disease condition. In related embodiment, the compound is Gingerenone A when R₁ is methyl in Formula I In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In a related aspect, the aquaporins are selected from the group consisting of AQP4 and AQP9. In a related aspect, modulating the expression of aquaporins improves water homeostasis in the subject. In another related embodiment, the diseases conditions are selected from the group consisting of neurodegenerative diseases, metabolic syndrome, cardiovascular diseases, cancer, inflammatory diseases, allergy, diabetes, insulin resistance, renal disorders, skin hydration, brain edema, immune system disorders, obesity, or liver steatosis. In a preferred embodiment, modulating the expression of aquaporins and associated biomarkers is useful for management of diseases and disorders selected from the group consisting of neurodegenerative diseases, diabetes, and insulin resistance. In another related embodiment, the effective dose of the compounds is 5-30 mg/kg bodyweight of the subject. In another preferred embodiment, the subject is a mammal. In a related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a method for maintaining or improving water homeostasis in the brain of a subject with a neurodegenerative condition, comprising a) identifying the subject with a neurodegenerative condition, b) administering an effective dose of a composition comprising compounds of Formula I to the subject. In a related embodiment of the invention, the compound is Gingerenone A., when R₁ is methyl in Formula I. In a related aspect, the improvement water homeostasis is brought about by modulating the expression of aquaporins (AQP). In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another preferred embodiment, improving water homeostasis results in improved cognitive and behavior patterns, improvement in neuronal plasticity and learning, reduction in inflammatory markers, improvement in neurotransmitter expression. In another related embodiment of the invention, the effective dose of the compounds is 5-20 mg/kg bodyweight of the subject. In another related embodiment of the invention, the degenerative condition is selected from the group consisting of, Alzheimer's disease, Parkinson's disease, Vascular dementia, neuromyelitis optica, amyotrophic lateral sclerosis, prion diseases, idiopathic-normal pressure hydrocephalus, Lewy body dementia, and fronto-temporal dementia. In a related aspect the subject is a mammal. In related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a composition comprising compounds of Formula I for use in maintaining or improving water homeostasis in the brain of a subject with a degenerative condition. In a related embodiment of the invention, the compound is Gingerenone A, when R₁ is methyl in Formula. I. In a related aspect, the improvement in water homeostasis is brought about by modulating the expression of aquaporins (AQP). In related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another preferred embodiment of the invention, improving water homeostasis results in improved cognitive and behavior patterns, improvement in neuronal plasticity and learning, reduction in inflammatory markers, improvement in neurotransmitter expression. In another related embodiment of the invention, the effective dose of the compounds is 5-20 mg/kg bodyweight of the subject. In another related embodiment of the invention, the degenerative condition is selected from the group consisting of, Alzheimer's disease, Parkinson's disease, Vascular dementia, neuromyelitis optica, Amyotrophic lateral sclerosis, prion diseases, idiopathic-normal pressure hydrocephalus, Lewy body dementia, and fronto-temporal dementia. In a related aspect, the subject is a mammal. In a related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses use of a composition comprising compounds of Formula I in maintaining or improving water homeostasis in the brain of a subject with a degenerative condition. In a related embodiment of the invention, the compound is Gingerenone A, when R₁ is methyl in Formula. I. In a related aspect, the improvement in water homeostasis is brought about by modulating the expression of aquaporins (AQP). In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another preferred embodiment of the invention, improving water homeostasis results in improved cognitive and behavior patterns, improvement in neuronal plasticity and learning, reduction in inflammatory markers, improvement in neurotransmitter expression. In another related embodiment of the invention, the effective dose of the compounds is 5-20 mg/kg bodyweight of the subject. In another related embodiment of the invention, the degenerative condition is selected from the group consisting of, Alzheimer's disease, Parkinson's disease, Vascular dementia, neuromyelitis optica, Amyotrophic lateral sclerosis, prion diseases, idiopathic-normal pressure hydrocephalus, Lewy body dementia, and fronto-temporal dementia. In a related aspect the subject is a mammal. In related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a method for therapeutic management of Alzheimer's disease in a subject comprising a) identifying the subject with Alzheimer's disease, b) administering an effective dose of a composition comprising compounds of Formula I to the subject. In a related embodiment of the invention, the compound is Gingerenone A, when R₁ is methyl in Formula L. In a related aspect, management of Alzheimer's disease is brought about by improving water homeostasis by increasing the expression of aquaporins, improving cognition and behavioural patterns, decreasing plaque formation by reducing β-amyloid content, decreasing neurofibrillary tangles by reducing phosphorylated tau formation, decreasing apolipoprotein E levels and reducing inflammatory markers. In related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment, the effective dose of the compounds is 5-20 mg/kg bodyweight of the subject. In a related aspect the subject is a mammal. In related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a composition comprising compounds of Formula I for use in the therapeutic management of Alzheimer's disease in a subject. In a related embodiment of the invention, the compound is Gingerenone A, when R₁ is methyl in Formula I. In a related aspect, management of Alzheimer's disease is brought about by improving water homeostasis by increasing the expression of aquaporins, improving cognition and behavioural patterns, decreasing plaque formation by reducing β-amyloid content, decreasing neurofibrillary tangles by reducing phosphorylated tau formation, decreasing apolipoprotein E levels and reducing inflammatory markers. In related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment, the effective dose of the compounds is 5-20 mg/kg bodyweight of the subject. In a related aspect the subject is a mammal. In related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses use of a composition comprising compounds of Formula I in the therapeutic management of Alzheimer's disease in a subject. In a related embodiment of the invention, the compound is Gingerenone A, when R₁ is methyl in Formula I. In a related aspect, management of Alzheimer's disease is brought about by improving water homeostasis by increasing the expression of aquaporins, improving cognition and behavioural patterns, decreasing plaque formation by reducing β-amyloid content, decreasing neurofibrillary tangles by reducing phosphorylated tau formation, decreasing apolipoprotein E levels and reducing inflammatory markers. In related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment, the effective dose of the compounds is 5-20 mg/kg bodyweight of the subject. In a related aspect, the subject is a mammal. In related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a method for therapeutic management of Parkinson's disease in a subject comprising a) identifying the subject with Parkinson's disease, b) administering an effective dose of a composition comprising compounds of Formula I to the subject. In a related embodiment of the invention, the compound is Gingerenone A, when R₁ is methyl in Formula I. In a related aspect, management of Parkinson's disease is brought about by improving water homeostasis by increasing the expression of aquaporins, increasing dopamine and other neurotransmitter levels, and reducing inflammatory markers. In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment, the effective dose of the compounds is 5-20 mg/kg bodyweight of the subject. In a related aspect the subject is a mammal. In a related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses a composition comprising compounds of Formula I for use in the therapeutic management of Parkinson's disease in a subject. In a related embodiment of the invention, the compound is Gingerenone A, when R₁ is methyl in Formula I. In a related aspect, management of Parkinson's disease is brought about by improving water homeostasis by increasing the expression of aquaporins, increasing dopamine and other neurotransmitter levels, and reducing inflammatory markers. In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment of the invention, the effective dose of the compounds is 5-20 mg/kg bodyweight of the subject. In a related aspect the subject is a mammal. In a related embodiment of the invention, the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In another most preferred embodiment, the invention discloses use of a composition comprising compounds of Formula I in the therapeutic management of Parkinson's disease in a subject. In a related embodiment of the invention, the compound is Gingerenone A, when R₁ is methyl in Formula I. In a related aspect, management of Parkinson's disease is brought about by improving water homeostasis by increasing the expression of aquaporins, increasing dopamine and other neurotransmitter levels, and reducing inflammatory markers. In a related embodiment of the invention, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof. In another preferred embodiment of the invention, the aquaporins are selected from the group consisting of AQP4 and AQP9. In another related embodiment, the effective dose of the compounds is 5-20 mg/kg bodyweight of the subject. In a related aspect the subject is a mammal. In a related embodiment of the invention the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

The preferred embodiments are further described in detail by means of the following examples. The examples are provided solely for illustration and are not to be construed as limitations of the present disclosure, as many variations thereof are possible without departing from the spirit and scope of the present disclosure.

### Example 1: Materials

From the compounds of Formula I, Gingerenone A, when R₁ is methyl of Formula I is evaluated for their ability to modify the expression of aquaporins in a diabetic animal model. Different methods and processes are available in the literature for the chemical synthesis of Gingerenone A. The compound can also be isolated from a plant source, preferably, but not limited to, Zingiber officinale, Alnus hirsuta, Alnus sibirica, Alnus glutinosa, Alnus formosana, Alnus japonica, Alnus nepalensis, Viscum cruciatum using conventionally known extraction processes.

### Example 2: Effect of Gingerenone A in the Management of Alzheimer's Disease

### Experimental Design

The abdominal region of the rat was carefully sanitized using 70% alcohol (v/v). Induction of Alzheimer's disease was done using aluminium chloride (AlCl₃). Stock solution of AlCl₃ is prepared by dissolving 400 mg of AlCl₃ in 10 ml of water to create a solution with a concentration of 40 mg/ml. Appropriate dosage of AlCl₃ is administered intraperitoneally using a 1 ml syringe. For the experimental groups, excluding the control, amnesia is induced through daily intraperitoneal injections of AlCl₃ at a dosage of 100 mg/kg from day 9 to day 17, following pre-treatment with the test drug. Half an hour after administering AlCl₃ on the 15th day, the Novel Object Recognition (NOR) test is performed. On the 17^{th} day, both the Elevated Plus Maze (EPM) and Morris Water Maze (MWM) tests are conducted to assess behavioural changes. At the study's conclusion, blood was collected from the retro-orbital plexus. Centrifugation of blood at 2500 rpm for 10 minutes and then separation of the serum was done for analysis of plasma biomarkers. Brains of the experimental animals were harvested for biochemical analyses and histopathological studies. The protocols used were as reported by Rahman et al. Epalrestat improves motor symptoms by reducing oxidative stress and inflammation in the reserpine induced mouse model of Parkinson's disease. Animal Model Exp Med. 2020; 3(1):9-21; Montarolo et al. NURR1 deficiency is associated to ADHD-like phenotypes in mice. Transl Psychiatry. 2019; 9(1):207. A test group of animals administered with Donepezil, a known drug for the management of Alzheimer's disease was used for comparison. Table 1 discloses the groups of rats used for the experimentation.

TABLE 1
Experimental groups

| Group | Treatment groups |
|---|---|
| I | Vehicle control (10 ml/kg, p.o.) |
| II | AD control (AlCl₃-100 mg/kg, i.p.) |
| III | Gingerenone A (5 mg/kg, p.o.) + |
| | AlCl₃ (100 mg/kg, i.p.) |
| IV | Gingerenone A (10 mg/kg, p.o.) + |
| | AlCl₃ (100 mg/kg, i.p.) |
| V | Gingerenone A (20 mg/kg, p.o.) + |
| | AlCl₃ (100 mg/kg, i.p.) |
| VI | Donepezil (5 mg/kg, p.o) + |
| | AlCl₃ (100 mg/kg, i.p.) |

### Assessment of Behavioural Parameters:

Novel object recognition (NOR)—Novel object recognition (NOR)—This test was performed as per reported protocol (Antunes M, Biala G. The novel object recognition memory: neurobiology, test procedure, and its modifications. Cognit. Process. 2012:13, 93-110). During the retrieval phase, the animal was allowed to explore the field for 10 min. The hippocampal functions were assessed through the recognition indices, which were measured according to the time spent by the animals to explore the familiar and new objects.

The test is a relatively fast and efficient means for testing learning and memory in an animal model. The test relies on only three sessions: one habituation session, one training session, and one test session and relies on rodents' natural proclivity for exploring novelty. This is a relatively low-stress, efficient test for memory in an animal model, and is appropriate for the detection of neuropsychological changes after the administration of test drugs (Lupetow L, Novel Object Recognition Test for the Investigation of Learning and Memory in Mice, J Vis Exp. 2017; (126): 55718). In the current study, the recognition index of animal groups with Alzheimer's were significantly reduced and the treatment with Gingerenone A effectively improved the scores (Table 2).

TABLE 2
Effect of Gingerenone A on behavioural
parameter in rats-NOR at 15th day

| | | % Recognition |
|---|---|---|
| Group | Treatment groups | Index |
| I | Vehicle control (10 ml/kg, p.o.) | 72.24 ± 2.83 |
| II | AD control (AlCl₃-100 mg/kg, i.p.) | 48.05 ± 2.67 |
| III | Gingerenone A (5 mg/kg, p.o.) + | 56.23 ± 2.35 |
| | AlCl₃ (100 mg/kg, i.p.) | |
| IV | Gingerenone A (10 mg/kg, p.o.) + | 65.54 ± 2.78** |
| | AlCl₃ (100 mg/kg, i.p.) | |
| \V | Gingerenone A (20 mg/kg, p.o.) + | 66.53 ± 3.20** |
| | AlCl₃ (100 mg/kg, i.p.) | |
| VI | Donepezil (5 mg/kg, p.o) + | 62.58 ± 2.74** |
| | AlCl₃ (100 mg/kg, i.p.) | |

Novel object recognition (NOR, n = 6, All the value were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

Novel object recognition (NOR), n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

Elevated plus maze (EPM)—Elevated plus maze (EPM)—This test was performed as per reported protocol (Bhuvanendran et al., Amelioration of cognitive deficit by Embelin in a Scopolamine-induced Alzheimer's disease-like condition in a rat model. Front Pharmacol. 2018:9(6):1-12 and Biradar et al., (2020) Experimental validation and network pharmacology evaluation to decipher the mechanism of action of Erythrina variegata L. bark against scopolamine-induced memory impairment in rats. Adv Tradit Med. 2020:26:1-4). EPM contains 2 closed and 2 open arms (four arms; equal dimensions of 50×10 cm). These 4 arms connect at central with a 10×10 cm square gap as an initial point. The transfer latency (TL) was recorded via stopwatch by keeping rats independently toward one side of the open arm. The EPM TL cut-off was set to 60 s throughout the study. In EPM, when the rat does not enter the closed arm, those rat were directed back into one of the closed arms, and 60 s TL was given. Index of memory improvement is measured as a drop of transfer Latency (TL) by test drug.

The elevated plus maze is a test that is widely used to assess the anti-anxiety effects of test substances. As the test measures open arm activity, an increase in open arm activity (duration and/or entries) reflects anti-anxiety behaviour (Walf et al., The use of the elevated plus maze as an assay of anxiety-related behavior in rodents, Nat Protoc. 2007; 2(2): 322-328). In the present study, inducing Alzheimer's in the animals increased their anxiety which was reduced by the administration on Gingerenone A (Table 3).

TABLE 3
Effect of Gingerenone A on behavioural parameter-EPM in rats

| | | EPM at 16^{th} day | EPM at 17^{th} day |
|---|---|---|---|
| | | Transfer Latency | Transfer Latency |
| Group | Treatment groups | (Sec) | (Sec) |
| I | Vehicle control (10 ml/kg, p.o.) | 17.19 ± 1.08 | 15.80 ± 0.58 |
| II | AD control (AlCl₃-100 mg/kg, | 32.28 ± 1.23 | 30.65 ± 1.45 |
| | i.p.) | | |
| III | Gingerenone A (5 mg/kg, p.o.) + | 25.70 ± 0.88 | 25.21 ± 1.04 |
| | AlCl₃ (100 mg/kg, i.p.) | | |
| IV | Gingerenone A (10 mg/kg, p.o.) + | 21.76 ± 1.03* | 19.37 ± 0.47* |
| | AlCl₃ (100 mg/kg, i.p.) | | |
| V | Gingerenone A (20 mg/kg, p.o.) + | 19.38 ± 0.87* | 18.59 ± 0.86** |
| | AlCl₃ (100 mg/kg, i.p.) | | |
| VI | Donepezil (5 mg/kg, p.o) + | 18.57 ± 0.68 | 18.22 ± 0.92** |
| | AlCl₃ (100 mg/kg, i.p.) | | |

Elevated plus maze (EPM), n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, ***P < 0.001 when compared to AD control group.

Elevated plus maze (EPM), n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, ***P<0.001 when compared to AD control group.

Morris water maze (MWM)—This test was performed as per protocol established by Ishola et al., Protective role of Spondiasmombin leaf and Cola acuminata seed extracts against scopolamine-induced cognitive dysfunction. Alexandria J Med. 2018; 54(1):27-39. MWM is a round water tank “diameter: 110 cm and height: 60 cm” consisting of a white surface and that is filled using opaque water (Temp. 26±2° C.) to a distance (depth) of 30 cm. The MWM tank circle is divided into 4 equal quadrants [North Quadrant 1, East Quadrant 2, West Quadrant 3, and South Quadrant 4]. Further, it consists of a 10 cm diameter platform in a constant position. Index of memory improvement is measured by the Escape Latency Time (ELT)—which is the time the animal takes to find the platform. The ELT of the individual rat was noted at the 60 s cut-off time.

The MWM plays an important role in the testing of learning and memory in rodent models of Alzheimer's Disease. It is used to test hippocampal-dependent learning, including acquisition of spatial memory and long-term spatial memory (Bromley-Brits et al., Morris water maze test for learning and memory deficits in Alzheimer's disease model mice, J Vis Exp., 2011 Jul. 20:(53):2920). In this study, learning was impaired in the Alzheimer's group. Administration Gingerenone A significantly improved learning and memory as observed by the water maze test results (Table 4).

TABLE 4
Effect of Gingerenone A on behavioural
parameter in rats-MWM at 17^{th} day

| | | Escape Latency |
|---|---|---|
| Group | Treatment groups | Time (Sec) |
| I | Vehicle control (10 ml/kg, p.o.) | 24.35 ± 3.54 |
| II | AD control (AlCl3-100 mg/kg, i.p.) | 47.65 ± 3.76 |
| III | Gingerenone A (5 mg/kg, p.o.) + | 34.47 ± 3.87* |
| | AlCl3 (100 mg/kg, i.p.) | |
| IV | Gingerenone A (10 mg/kg, p.o.) + | 27.73 ± 2.18*** |
| | AlCl3 (100 mg/kg, i.p.) | |
| V | Gingerenone A (20 mg/kg, p.o.) + | 27.82 ± 2.63** |
| | AlCl3 (100 mg/kg, i.p.) | |
| VI | Donepezil (5 mg/kg, p.o) + | 26.96 ± 2.58*** |
| | AlCl3 (100 mg/kg, i.p.) | |

Morris water maze (MWM), n = 6, All the values were expressed in Mean ± SEM (n = 6). The statistical analysis was carried out uing one way ANOVA. Significant after analysis of variance (ANOVA) followed by Dunnett multiple comparison test.
*P < 0.05, **P < 0.01, ***P < 0.001 when compared to AD control group.

Morris water maze (MWM), n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, ***P<0.001 when compared to AD control group.

### Evaluation of Protein Expression (AQP4, AChE, BDNF) in a Brain Homogenate

Aquaporin-4 (AQP4) is a channel protein that plays a fundamental role in glymphatic system, a pathway for fluid exchange in the central nervous system, as well as a central figure in the pathophysiology of neurodegenerative diseases such as Alzheimer's disease (AD) and frontotemporal dementia. It plays an important role in maintaining cerebral fluid homeostasis by acting as a water channel protein. The AQP4 not only enables water permeability through the blood-brain barrier and also regulates water exchange between perivascular spaces and the rest of the glymphatic system (Szczygielski et al., Cerebral Microcirculation, Perivascular Unit, and Glymphatic System: Role of Aquaporin-4 as the Gatekeeper for Water Homeostasis, Front. Neurol. 12:767470, doi: 10.3389/fneur.2021.767470). Further, Astrocytes are known to maintain extracellular potassium concentrations in the brain by a process termed “potassium siphoning”. Changes in potassium channel function alters brain homeostasis leading to brain impairment. Water movement via the AQP4 water channel localized to astrocyte end-feet maintains osmotic balance and promotes effective potassium siphoning (Verkman A S, Aquaporins in Clinical Medicine, Annu. Rev. Med. 2012. 63:303-16). Thus, increasing AQP4 levels maintains effective potassium siphoning and prevents or delays neurodegeneration. In the present study, the levels were determined using ELISA kit (Biogenuix Medsystems Pvt. Ltd.). AQP4 levels were significantly reduced in Alzheimer's (AD) controls and administration of Gingerenone A increased AQP4 expression (FIG. 1).

Acetyl cholinesterase—A common feature in the Alzheimer's disease (AD) brain is the presence of acetylcholinesterase (AChE) which is commonly associated with 0-amyloid plaques and neurofibrillary tangles (NFT). Acetylcholinesterase (AChE) is a key enzyme in the cholinergic nervous system. During the progression of AD, many different types of neurons deteriorate, although there is a profound loss of forebrain cholinergic neurons, which is accompanied by a progressive decline in acetylcholine. Current AD therapy is mostly based on inhibitors of AChE (AChE-I), which enhance cholinergic transmission.

At the conclusion of the study, the rats were euthanized by decapitation, and their brains were swiftly removed and placed in ice-cold saline solution. The cortex and hippocampus were carefully isolated and submerged in an ice-cold water container to halt enzymatic reactions. The levels of acetylcholinesterase (AChE) and butyrylcholinesterase (BChE) in the cortex and hippocampus regions of the brain were determined using Ellman's method from 1961. Approximately 100 mg of the isolated brain sections were weighed and homogenized in phosphate buffer (0.1 M, pH 8.0). To 0.4 mL of the homogenate, the following components were added: 2.6 mL of phosphate buffer (0.1 M, pH 8.0), 100 μL of 10 mM 5,5-dithio-bis-(2-nitrobenzoic acid) (DTNB), and 20 μL of acetylthiocholine iodide (AChI) solution (0.075 M). The mixture was thoroughly mixed. The absorbance was then measured at 412 nm for a duration of 5 minutes. The rate of substrate hydrolysis was calculated as moles of substrate hydrolyzed per minute per gram of tissue (Ahmed et al., Acetylcholinesterase activity in the brain of alloxan diabetic albino rats: Presence of an inhibitor of this enzyme activity in the cerebral extract. Int J Diabetes Dev Ctries. 2009; 29(4):174-7).

Gingerenone A significantly inhibited Acetyl cholinesterase thereby increasing the neurotransmitter acetyl choline levels and correspondingly increased cholinergic transmission (Table 5).

TABLE 5
Effect of Gingerenone A on expression of Acetylcholinesterase

| | | Ach E |
|---|---|---|
| | | (10⁻⁶ /mol/g |
| Group | Treatment groups | of tissue) |
| I | Vehicle control (10 ml/kg, p.o.) | 3.63 ± 0.21 |
| II | AD control (AlCl₃-100 mg/kg, i.p.) | 14.02 ± 0.62 |
| III | Gingerenone A (5 mg/kg, p.o.) + | 11.76 ± 0.93 |
| | AlCl₃ (100 mg/kg, i.p.) | |
| IV | Gingerenone A (10 mg/kg, p.o.) + | 8.77 ± 0.49** |
| | AlCl₃ (100 mg/kg, i.p.) | |
| V | Gingerenone A (20 mg/kg, p.o.) + | 6.87 ± 0.34** |
| | AlCl₃ (100 mg/kg, i.p.) | |
| VI | Donepezil (5 mg/kg, p.o) + | 4.25 ± 0.48** |
| | AlCl₃ (100 mg/kg, i.p.) | |

n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

Brain-derived neurotrophic factors (BDNFs) play an important role in maintaining synaptic plasticity in learning and memory. Depletion of BDNF has been linked with Aβ accumulation, tau phosphorylation, neuroinflammation and neuronal apoptosis. Considering AD as a synaptic disorder, BDNF has attracted increasing attention as a potential diagnostic biomarker and a therapeutical molecule for AD. In the current study, BDNF levels were determiner using ELISA kit (Krishgen Biosystems, India). Gingerenone A significantly increased the levels of BDNF (Table 6).

TABLE 6
Effect of Gingerenone A on the expression of BDNF

| | | BDNF |
|---|---|---|
| | Treatment groups | (pg/ml) |
| I | Vehicle control (10 ml/kg, p.o.) | 45.32 ± 2.34 |
| II | AD control (AlCl₃-100 mg/kg, i.p.) | 22.78 ± 1.25 |
| III | Gingerenone A (5 mg/kg, p.o.) + | 29.36 ± 1.09 |
| | AlCl₃ (100 mg/kg, i.p.) | |
| IV | Gingerenone A (10 mg/kg, p.o.) + | 36.07 ± 1.54* |
| | AlCl₃ (100 mg/kg, i.p.) | |
| V | Gingerenone A (20 mg/kg, p.o.) + | 38.76 ± 2.05** |
| | AlCl₃ (100 mg/kg, i.p.) | |
| VI | Donepezil (5 mg/kg, p.o) + | 31.51 ± 2.07** |
| | AlCl₃ (100 mg/kg, i.p.) | |

n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

### Evaluation of Protein Biomarkers (Amyloid β, Apolipoprotein E, Tau) in Plasma

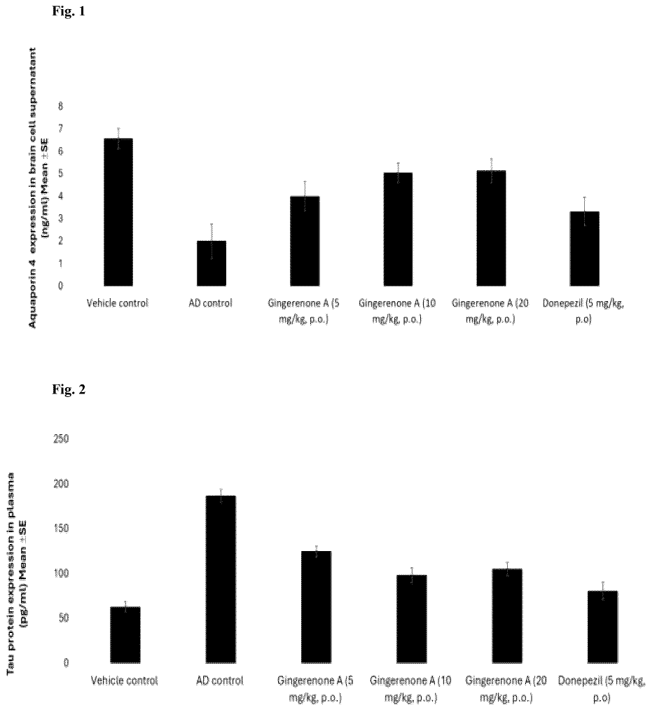

The levels of Tau, β-amyloid (1-42) and Apolipoprotein E were estimated. using ELISA kit (Krishgen Biosystems, India). All the protein biomarkers of AD were elevated in the AD control group. Gingerenone A significantly reduced the levels of β-amyloid (Table 7), Tau (FIG. 2) and Apolipoprotein E (Table 8).

TABLE 7
Effect of Gingerenone A on expression of amyloid β

| Group | Treatment groups | β-amyloid (pg/ml) |
|---|---|---|
| I | Vehicle control (10 ml/kg, p.o.) | 4.98 ± 0.023 |
| II | AD control (AlC13-100 mg/kg, i.p.) | 18.93 ± 0.054 |
| III | Gingerenone A (5 mg/kg, p.o.) + | 10.74 ± 0.035* |
| | AlCl3 (100 mg/kg, i.p.) | |
| IV | Gingerenone A (10 mg/kg, p.o.) + | 8.04 ± 0.023** |
| | AlCl3 (100 mg/kg, i.p.) | |
| V | Gingerenone A(20 mg/kg, p.o.) + | 7.88 ± 0.065** |
| | AlCl3 (100 mg/kg, i.p.) | |
| VI | Donepezil (5 mg/kg, p.o) + | 8.02 ± 0.043** |
| | AlCl3 (100 mg/kg, i.p.) | |

n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

TABLE 8
Effect of Gingerenone A on expression of Apolipoprotein E

| | | Apolipoprotein E |
|---|---|---|
| Group | Treatment groups | (ng/ml) |
| I | Vehicle control (10 ml/kg, p.o.) | 6.66 ± 0.076 |
| II | AD control (AlCl₃-100 mg/kg, i.p.) | 9.78 ± 0.056 |
| III | Gingerenone A (5 mg/kg, p.o.) + | 7.47 ± 0.054 |
| | AlCl₃ (100 mg/kg, i.p.) | |
| IV | Gingerenone A A(10 mg/kg, p.o.) + | 7.08 ± 0.076* |
| | AlCl₃ (100 mg/kg, i.p.) | |
| V | Gingerenone A(20 mg/kg, p.o.) + | 6.84 ± 0.034** |
| | AlCl₃ (100 mg/kg, i.p.) | |
| VI | Donepezil (5 mg/kg, p.o) + | 6.96 ± 0.065** |
| | AlCl₃ (100 mg/kg, i.p.) | |

n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

### Effect of Gingerenone A on Inflammatory Markers

The inflammatory markers Fibrinogen, IL-1β were estimated using ELISA kit (Krishgen Biosystems, India).

The major physiological function of fibrinogen is the formation of fibrin that binds together platelets and some plasma proteins in a haemostatic plug. In pathological situations, the network entraps large numbers of erythrocytes and leukocytes forming a thrombus that may occlude a blood vessel. Fibrinogen binds to Aβ, which intensifies inflammation in the AD brain and accelerates the decline of cognitive function in AD patients. Similarly, IL-1β is intimately involved in elaboration of acute neuroinflammatory processes in vivo. Exposure of the rodent brain to IL-1β elicits rapid, robust activation of both astrocytes and microglia. In addition to its role in elaboration of neuroinflammation and leukocyte recruitment, local expression of IL-1 has been implicated in impairment of hippocampal dependent memory processing. In the current study, Gingerenone A significantly reduced the inflammatory markers fibrinogen and IL-1β (Table 9).

TABLE 9
Effect of Gingerenone A on Inflammatory
markers Fibrinogen, IL-1β

| | | Fibrinogen | IL-1β |
|---|---|---|---|
| Group | Treatment groups | (ng/ml) | (pg/ml) |
| I | Vehicle control (10 ml/kg, p.o.) | 12.76 ± 0.065 | 54.96 |
| II | AD control (AlCl₃-100 mg/kg, | 27.66 ± 0.212 | 106.94 |
| | i.p.) | | |
| III | Gingerenone A (5 mg/kg, p.o.) + | 22.80 ± 0.738 | 97.92 |
| | AlCl₃ (100 mg/kg, i.p.) | | |
| IV | Gingerenone A(10 mg/kg, p.o.) + | 19.05 ± 0.232* | 84.90* |
| | AlCl₃ (100 mg/kg, i.p.) | | |
| V | Gingerenone A (20 mg/kg, p.o.) + | 16.65 ± 0.431** | 80.65** |
| | AlCl₃ (100 mg/kg, i.p.) | | |
| VI | Donepezil (5 mg/kg, p.o) + | 16.77 ± 0.053** | 62.16** |
| | AlCl₃ (100 mg/kg, i.p.) | | |

n = 6, All the values were expressed in Mean ± SEM (n = 6). The statistical analysis was carried out using one way ANOVA. Significant after analysis of variance (ANOVA) followed by Dunnett multiple comparison test.
*P < 0.05, **P < 0.01, when compared to AD control group.

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, when compared to AD control group.

### Analysis of Brain Histopathology

The brain histopathology was done for all the experimental groups. The brain tissue was fixed in 10% buffered neutral formalin solution. The tissue was embedded in paraffin, sectioned at 5 m stained with hematoxylin and eosin (H&E). The sections were examined under light microscope to study the architecture of the brain tissue, and then photomicrographs were taken (Fischer A H, Jacobson K A, Rose J, Zeller R. Hematoxylin and eosin staining of tissue and cell sections. CSH Protoc. 2008:1;pdb.prot4986. doi: 10.1101/pdb.prot4986).

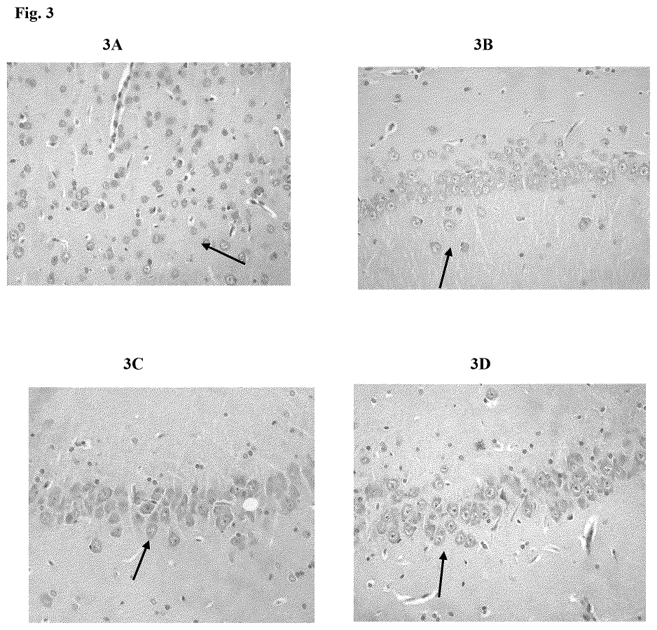

The frontal cortex section of the vehicular control (FIG. 3) showed intact neurocytes having round to oval vesicular nucleus having abundant cytoplasm along with neuroglial cells, granular cells and intact blood vessels (FIG. 3A) along with compact layers of pyramidal cells having vesicular nuclei and moderate to abundant cytoplasm without shrinkage and necrosis in all of CA1 (FIG. 3B), CA2 (FIG. 3C) and CA3 (FIG. 3D) regions.

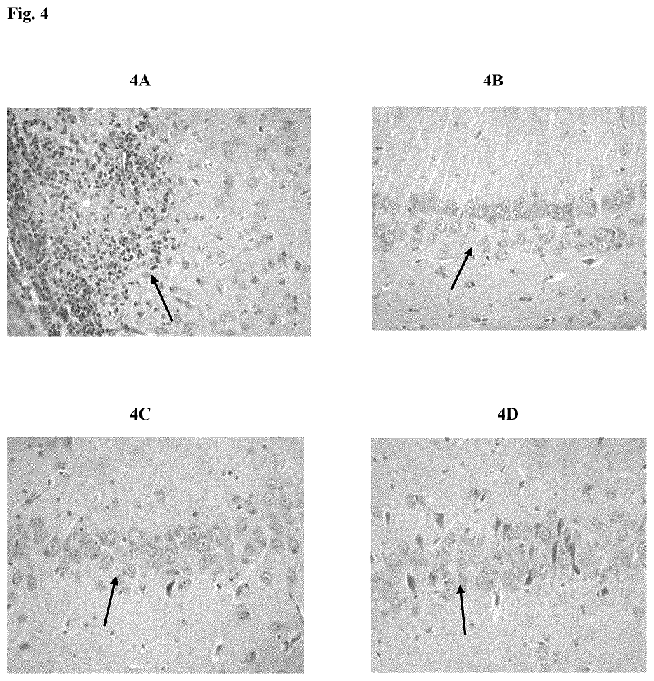

Induction of Alzheimer's disease by administration of AlCl₃ significantly changed the brain pathology with the frontal cortex of AD group (FIG. 4) showing intact pyramidal cells having round to oval vesicular nucleus with abundant cytoplasm, neuroglial cells, granular cells and intact blood vessels along with few degenerated cells [moderate] with dystrophic changes with dense inflammation (FIG. 4A). The hippocampus showed intact architecture consisting of Cornu Ammonis [CA] and dentate gyrus with areas of cell loss of pyramidal cells having vesicular nuclei and moderate cytoplasm with focal shrinkage of some cells in all of CA1 (FIG. 4B), CA2 (FIG. 4C) and CA3 (FIG. 4D) regions.

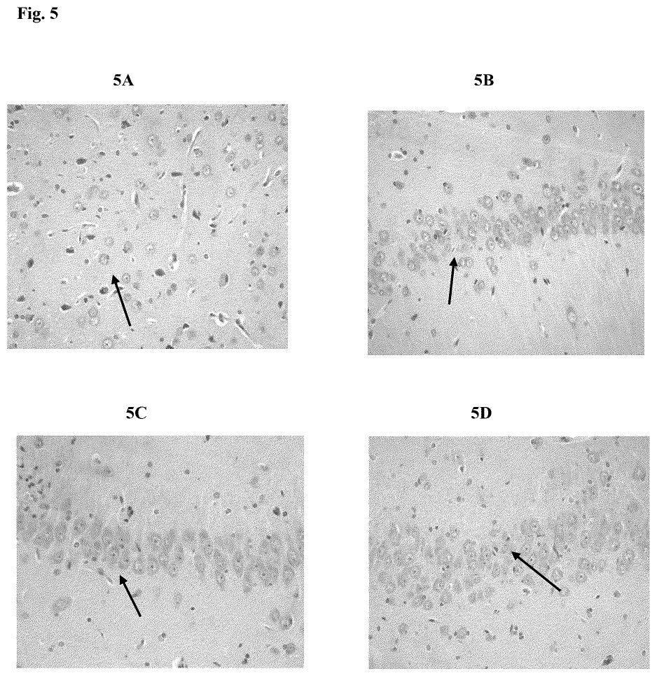

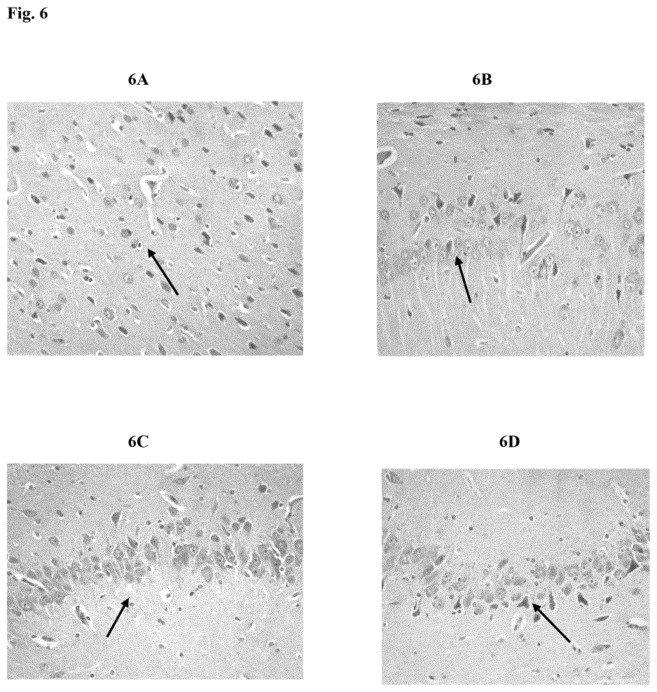

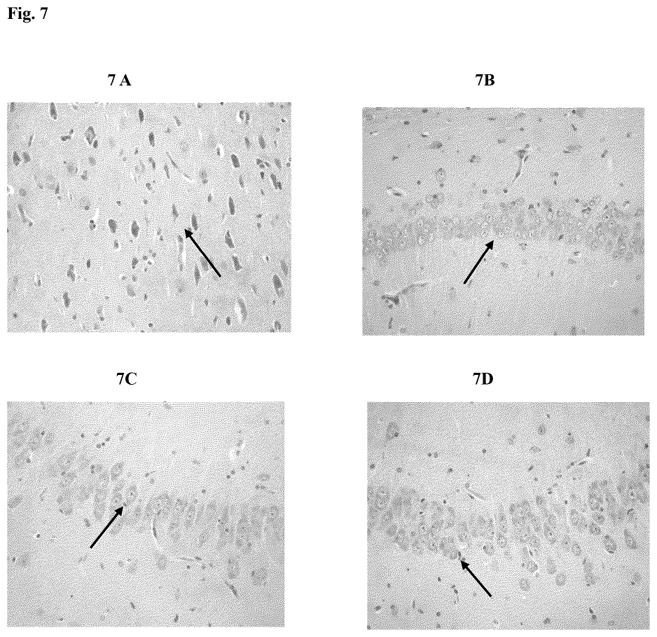

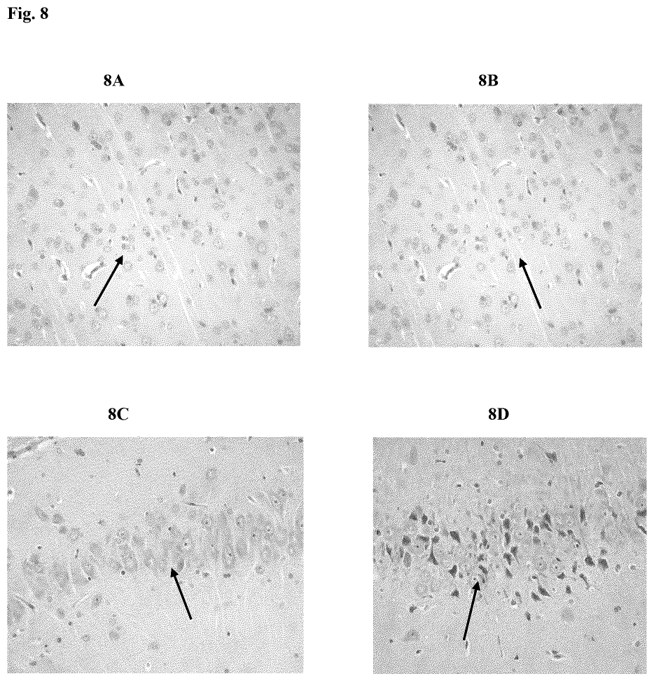

Administration of Gingerenone A at 5 mg/kg (FIG. 5), 10 mg/kg (FIG. 6) and 20 mg/kg (FIG. 7) improved the cellular structure with frontal cortex showing intact pyramidal cells having round to oval vesicular nucleus with abundant cytoplasm, neuroglial cells, granular cells and intact blood vessels with degenerated cells [mild to moderate] with dystrophic changes having shrunken and hyperchromatic nuclei and without inflammation (FIG. 5A, FIG. 6A, FIG. 7A) and hippocampus showed intact architecture consisting of Cornu Ammonis [CA] and dentate gyrus with compact layers of pyramidal cells having vesicular nuclei and moderate cytoplasm without shrinkage and necrosis in most of CA1 (FIG. 5B, FIG. 6B, FIG. 7B), CA2 (FIG. 5C, FIG. 6C, FIG. 7C) and CA3 (FIG. 5D, FIG. 6D, FIG. 7D) regions. Donepezil control group (FIG. 8) also exhibited good frontal cortex with intact pyramidal cells having round to oval vesicular nucleus with abundant cytoplasm, neuroglial cells, granular cells and intact blood vessels with degenerated cells [focal] with dystrophic changes having shrunken and hyperchromatic nuclei (FIG. 8A) and hippocampus with intact architecture consisting of Cornu Ammonis [CA] and dentate gyrus with compact layers of small pyramidal cells having vesicular nuclei and moderate cytoplasm without shrinkage and necrosis with some degenerated cells with mild dystrophic changes having shrunken and hyperchromatic nuclei in CA1 (FIG. 8B), CA2 (FIG. 8C) and CA3 (FIG. 8D) regions respectively.

Overall administration of Gingerenone A resulted in improvement in cognition, learning and memory by improving the behavioural patterns, improved the water homeostasis in brain by enhancing AQP4, increased cholinergic transmission by inhibiting acetyl cholinesterase, Showed recovery in maintaining brain plasticity in memory & learning by enhancing BDNF levels, decreased the biomarkers involved in the pathogenicity of Alzheimer's disease (Tau, amyloid and Apo E), decreased inflammation and showed recovery in brain pathology, signifying that Gingerenone A is very effective for the management of Alzheimer's disease and dementia.

### Example 3: Effect of Gingerenone A in the Management of Parkinson's Disease

### Experimental Design

The induction of Parkinson's disease and measurement of other parameters were done as per the reported protocol (Santos et al., Cognitive, motor and tyrosine hydroxylase temporal impairment in a model of parkinsonism induced by reserpine. Behav Brain Res. 2013; 253:68-77 and Shishehbore et al. A novel kinetic spectrophotometric method for the determination of dopamine in biological and pharmaceutical samples. J Chem. 2013; 2013:1-6). Induction of Parkinson's disease was done by administering Resperine, which inhibits the monoamine vesicular transporters, thus decreasing the levels of monoamine neurotransmitters in the brain. Following pretreatment for 14 days with Gingerenone A, the rat groups received subcutaneous injections of reserpine (4 mg/kg) for the final five days of the treatment regimen. Resperine (5 mg) was dissolved in 1 ml of 1% acetic acid and then diluted tenfold with distilled water to achieve a final concentration of 500 μg/ml. The normal control group received saline with 1% acetic acid for 14 days, administered from day 10 to day 14. Thirty minutes following the administration of reserpine on the 14th day, behavioural assessments were conducted using the Open Field test, Y Maze, and Catalepsy tests to evaluate changes. Rats administered with Carbidopa, a known drug for the management of Parkinson's disease was also used for comparison. Upon completion of the study, brains were harvested for biochemical analyses and histopathological studies. The experimental group is depicted in table 10.

TABLE 10
Experimental group

| Group | Treatment groups |
|---|---|
| I | Vehicle control (10 ml/kg, p.o.) |
| II | Parkinson's disease (PKD) control (Reserpine-4 mg/kg, s.c.) |
| III | Gingerenone A (5 mg/kg, p.o.) + |
| | Reserpine (4 mg/kg, s.c.) |
| IV | Gingerenone A (10 mg/kg, p.o.) + |
| | Reserpine (4 mg/kg, s.c.) |
| V | Gingerenone A (20 mg/kg, p.o.) + |
| | Reserpine (4 mg/kg, s.c.) |
| VI | Carbidopa (25 mg/kg,p.o.) + |
| | Reserpine (4 mg/kg, s.c.) |

As explained in example 2 (Alzheimer's disease model), behavioural parameters (open filed test, Y maze test and Catalepsy test), biomarkers (Aquaporin 9 and dopamine) and brain histopathology were done to assess the effectiveness of Gingerenone A.

### Assessment of Behavioural Parameters

Open filed test—The test was conducted as per protocol described by Saleem et al., Pharmacological Potential of the Standardized Methanolic Extract of Prunus armeniaca L. in the Haloperidol-Induced Parkinsonism Rat Model. Evid Based Complement Alternat Med. 2022:29; 3697522. doi: 10.1155/2022/3697522. This test was conducted to assess the locomotor and exploratory behaviors of the experimental rats. A square-shaped wooden box measuring 100 cm in width, 100 cm in diameter, and 45 cm in height was constructed using plywood material, painted white, with a black-lined floor divided into 25 blocks. The rats were placed in the center of the field for a duration of 5 minutes and allowed to explore the box freely. The number of squares crossed, both centrally and horizontally, along with the total number of crossings, instances of defecation, freezing behavior, and postures were recorded during this period. Normal animals readily and quickly acclimate to the chamber and try to explore the center area of the squares. Impaired animals spend significantly less time in the open area and more time closer to the walls (Carter and Shieh, Animal behaviour, In: Guide to Research Techniques in Neuroscience (Second Edition) 2015, Pages 39-71, Academic Press). Induction of Parkinsonism significantly impaired the locomotory and exploratory behaviors of the animals and administration of Gingerenone A significantly improved the behavior (Table 11).

TABLE 11
Effect of Gingerenone A on behavioural parameter in rats-open
field test

| | | Number of | Number of | |
|---|---|---|---|---|
| | Treatment | squares | central | |
| Group | groups | crossed | explorations | Freezing (Sec) |
| I | Vehicle | 33.25 ± 3.54 | 21.57 ± 1.04 | 0.00 ± 0.0 |
| | control (10 | | | |
| | ml/kg, p.o.) | | | |
| II | PKD control | 14.62 ± 1.96 | 8.56 ± 0.76 | 45.29 ± 2.34 |
| | (Reserpine-4 | | | |
| | mg/kg, s.c.) | | | |
| III | Gingerenone | 21.62 ± 2.47 | 12.57 ± 1.05 | 15.29 ± 0.35** |
| | A (5 mg/kg, | | | |
| | p.o.) + | | | |
| | Reserpine (4 | | | |
| | mg/kg, s.c.) | | | |
| IV | Gingerenone | 27.45 ± 2.06** | 17.34 ± 1.20** | 10.38 ± 0.62*** |
| | A (10 mg/kg, | | | |
| | p.o.) + | | | |
| | Reserpine (4 | | | |
| | mg/kg, s.c.) | | | |
| V | Gingerenone | 26.26 ± 2.27** | 17.27 ± 0.98** | 9.02 ± 0.45*** |
| | A (20 mg/kg, | | | |
| | p.o.) + | | | |
| | Reserpine (4 | | | |
| | mg/kg, s.c.) | | | |
| VI | Carbidopa (25 | 31.80 ± 3.18** | 20.25 ± 1.03** | 9.63 ± 0.60*** |
| | mg/kg,p.o.) + | | | |
| | Reserpine (4 | | | |
| | mg/kg, s.c.) | | | |

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, ***P<0.01, when compared to PKD control group.

Y-maze test—In the Y-maze test, spontaneous alterations serve as an indicator of working short-term memory.

The maze consisted of a triangular central area with three equilateral arms labeled A, B, and C, each measuring 35 cm in length, 25 cm in height, and 10 cm in width. During the test, each rat was placed in the central area, facing one of the arms. An entry was scored when all four paws of the rat entered an arm. Spontaneous alterations in behavior were defined as consecutive entries into different arms (e.g., ABC, BCA, or CAB, but not CAC). The maximum number of spontaneous alterations (total number of arms entered) and the percentage of spontaneous alterations (actual number of alterations divided by the maximum alterations, multiplied by 100) were calculated for each rat (Kraeuter et al., The Y-Maze for Assessment of Spatial Working and Reference Memory in Mice. Methods Mol Biol. 2019; 1916:105-111). Animals in PKD group showed limited number of entries in the different arms with lesser number of alteration, a mark of reduced working memory. Administration of Gingerenone A significantly increase the working memory with increase in entries in the different arms with more number of alterations (Table 12)

TABLE 12
Effect of Gingerenone A on behavioral parameter in rats-Y maze
test

| | | No of arm | Spontaneous |
|---|---|---|---|
| Group | Treatment groups | entries | alterations (%) |
| I | Vehicle control | 14.76 ± 2.87 | 31.07 ± 1.67 |
| | (10 ml/kg, p.o.) | | |
| II | PKD control | 6.25 ± 0.68 | 10.88 ± 0.39 |
| | (Reserpine-4 mg/kg, s.c.) | | |
| III | Gingerenone A (5 mg/kg, | 9.37 ± 0.29 | 21.43 ± 1.08* |
| | p.o.) + | | |
| | Reserpine (4 mg/kg, s.c.) | | |
| IV | Gingerenone A(10 mg/kg, | 11.57 ± 0.92* | 26.87 ± 1.05** |
| | p.o.) + | | |
| | Reserpine (4 mg/kg, s.c.) | | |
| V | Gingerenone A(20 mg/kg, | 12.09 ± 1.39** | 26.21 ± 0.98** |
| | p.o.) + | | |
| | Reserpine (4 mg/kg, s.c.) | | |
| VI | Carbidopa (25 mg/kg,p.o.) + | 12.58 ± 1.04** | 27.31 ± 1.56** |
| | Reserpine (4 mg/kg, s.c.) | | |

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01. when compared to PKD control group.

Catalepsy Test—The catalepsy test was performed as per protocol described by Garabadu D, Agrawal N. Naringin exhibits neuroprotection against rotenone-induced neurotoxicity in experimental rodents. NeuroMol Med. 202022(2):314-330. Catalepsy in rats' manifests as an unresponsiveness to external stimuli accompanied by muscle rigidity. During the catalepsy test, rats were positioned on a wooden bar elevated 3-9 cm above the surface, with their forelimbs resting on it after administration of reserpine. The time taken for the rats to correct their posture was recorded as an indicator of catalepsy. Catalepsy ceased either when the rats climbed up the bar or when their forelimbs touched the floor. Observations were made at 30, 60, and 120 minutes after administration. All assessments were conducted in a quiet environment at a temperature of 23-25 degrees Celsius, with a maximum observation time of 5 minutes per trial. The scoring for catalepsy was as follows:
Score 0: Rats exhibited normal movement upon placement on the table.
Score 0.5: Rats responded normally when pushed or touched.
Score 2: Rats were unable to correct their posture within 10 seconds; 1 point was assigned for each affected paw.

The animal in the PKD group exhibited more muscle rigidity as evidenced by higher catalepsy score (Table 13) and administration of Gingerenone A significantly improved muscle rigidity.

TABLE 13
Effect of Gingerenone A on behavioural parameter in rats-
catalepsy test

| | Treatment | | | |
|---|---|---|---|---|
| Group | groups | At 30 min | At 60 min | At 120 min |
| I | Vehicle | 0.00 ± 0.00 | 0.00 ± 0.00 | 0.00 ± 0.00 |
| | control (10 | | | |
| | ml/kg, p.o.) | | | |
| II | PKD control | 5.34 ± 0.36 | 5.08 ± 0.41 | 5.40 ± 0.32 |
| | (Reserpine-4 | | | |
| | mg/kg, s.c.) | | | |
| III | Gingerenone | 2.46 ± 0.15 | 2.52 ± 0.54** | 2.43 ± 0.70** |
| | A (5 mg/kg, | | | |
| | p.o.) + | | | |
| | Reserpine (4 | | | |
| | mg/kg, s.c.) | | | |
| IV | Gingerenone | 1.76 ± 0.11** | 1.72 ± 0.73** | 1.71 ± 0.75** |
| | A (10 mg/kg, | | | |
| | p.o.) + | | | |
| | Reserpine (4 | | | |
| | mg/kg, s.c.) | | | |
| V | Gingerenone | 1.58 ± 0.16*** | 1.55 ± 0.32*** | 1.50 ± 0.82*** |
| | A (20 mg/kg, | | | |
| | p.o.) + | | | |
| | Reserpine (4 | | | |
| | mg/kg, s.c.) | | | |
| VI | Carbidopa (25 | 1.52 ± 0.42*** | 1.60 ± 0.70*** | 1.45 ± 0.70*** |
| | mg/kg,p.o.) + | | | |
| | Reserpine (4 | | | |
| | mg/kg, s.c.) | | | |

n=6, All the values were expressed in Mean±SEM (n=6). The statistical analysis was carried out using one way ANOVA. Significant after analysis of variance (ANOVA) followed by Dunnett multiple comparison test. *P<0.05, **P<0.01, ***P<0.01, when compared to PKD control group.

### Evaluation of Biochemical Markers (AQP9, Dopamine)

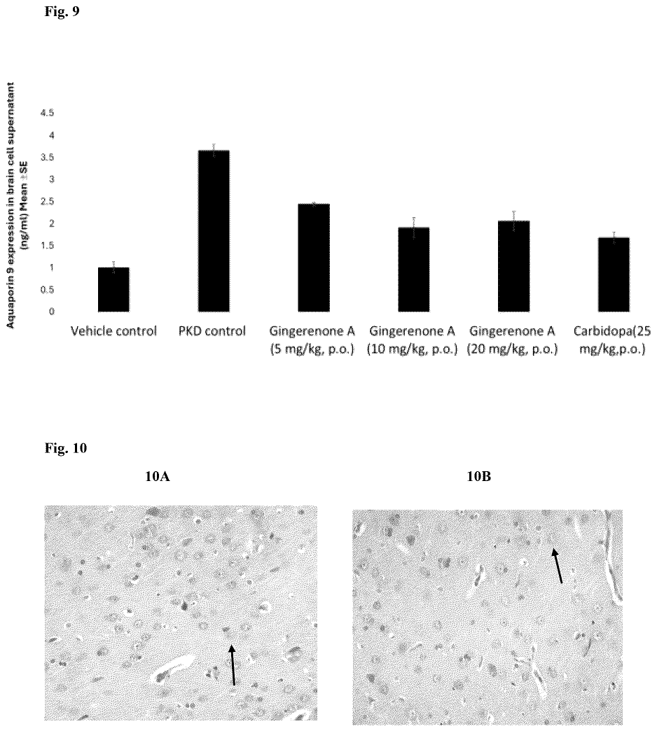

Aquaporin 9 (AQP9)—An intriguing link between AQP9 and PKD in reported in the literature. In the brain, water and solute channel is expressed in astrocytes, brain stem catecholaminergic neurons, and in subsets of midbrain dopaminergic and hypothalamic neurons. As to the functional role of AQP9, there is a rich literature indicating that this aquaporin is involved in peripheral inflammatory responses. AQP9 plays a proinflammatory role in the brain and evidence indicate that deletion of AQP9 significantly suppressed the inflammatory response to the parkinsonian toxin 1-methyl-4-phenylpyridinium (MPP+)—a toxin that promotes a strong inflammatory response in brain (Zahl et al., Aquaporin-9 in the Brain Inflammatory Response: Evidence from Mice Injected with the Parkinsonogenic Toxin MPP+, Biomolecules. 2023 April; 13(4): 588). Increased mitochondrial AQP9 in dopaminergic neurons relates to their vulnerability in PKD. In the present investigation, AQP9 levels were increased in PKD control group signifying increase in inflammation, and administration of Gingerenone A inhibited AQP9 expression thereby supressing inflammation (FIG. 9)

Dopamine—Studies have shown that symptoms of Parkinson's develop in patients with an 80 percent or greater loss of dopamine-producing cells in the substantia nigra. Normally, dopamine operates in a delicate balance with other neurotransmitters to help coordinate the millions of nerve and muscle cells involved in movement. Neurons of the substantia nigra communicate with neurons of the basal ganglia by liberating the neurotransmitter dopamine (DA). Such an interaction at the biochemical level is responsible for the fine tuning of the movements. The proposed method for the estimation of dopamine is based on inhibitory effect of dopamine on the oxidation of thionine by bromate in acidic media. The change in absorbance was recorded spectrophotometrically at 601 nm (Shishehbore et al., Novel Kinetic Spectrophotometric Method for the Determination of Dopamine in Biological and Pharmaceutical Samples. 2013. Article ID 819460, https://doi.org/10.1155/2013/819460). In the present study, dopamine levels were significantly reduced in the PKD group, and the levels increased by the administration of Gingerenone A (Table 14).

TABLE 14
Effect of Gingerenone A on expression of Dopamine in brain

| Group | Treatment groups | Dopamine (μg/ml) |
|---|---|---|
| I | Vehicle control | 0.159 ± 0.013 |
| | (10 ml/kg, p.o.) | |
| II | PKD control | 0.108 ± 0.028 |
| | (Reserpine-4 mg/kg, s.c.) | |
| III | Gingerenone A (5 mg/kg, p.o.) + | 0.124 ± 0.007 |
| | Reserpine (4 mg/kg, s.c.) | |
| IV | Gingerenone A(10 mg/kg, p.o.) + | 0.142 ± 0.011** |
| | Reserpine (4 mg/kg, s.c.) | |
| V | Gingerenone A(20 mg/kg, p.o.) + | 0.141 ± 0.011*** |
| | Reserpine (4 mg/kg, s.c.) | |
| VI | Carbidopa (25 mg/kg,p.o.) + | 0.148 ± 0.037*** |
| | Reserpine (4 mg/kg, s.c.) | |

n=6, All the values were expressed in Mean±SEM (n=6). The statistical analysis was carried out using one way ANOVA. Significant after analysis of variance (ANOVA) followed by Dunnett multiple comparison test. **P<0.01, ***P<0.01, when compared to PKD control group.

### Analysis of Brain Histopathology

The brain histopathology was done for all the experimental groups as per protocol described in example 2.

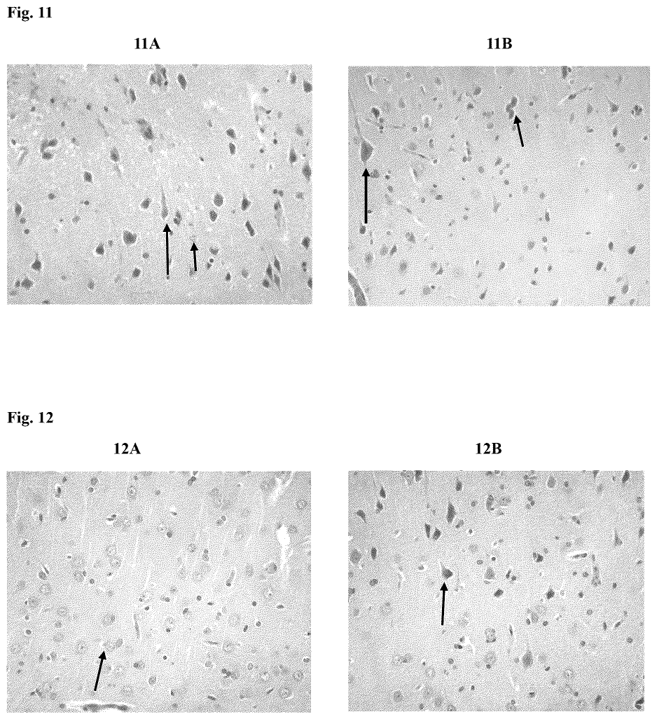

Histopathology sections from substantia nigra of Vehicular control (FIG. 10) showed predominantly neuronal cells (FIG. 10A—Arrow) within normal limits having bland nucleus with mild basophilic cytoplasm [FIG. 10B—Arrow]. The blood vessels appeared intact with normal neuropils. The sections from substantia nigra of PKD control (FIG. 11) showed intense degenerative neuronal cells having variable darkly stained nucleus [FIG. 11B—Short Arrow] with mild basophilic cytoplasm, neurofibrillary tangles [FIG. 11A—Long Arrow], extracellular neuromelanin [FIG. 11A—Short Arrow], reactive microgliosis, Lewy body [FIG. 11B—Long Arrow] and moderate inflammation. The neurophils was shown as vacuoles.

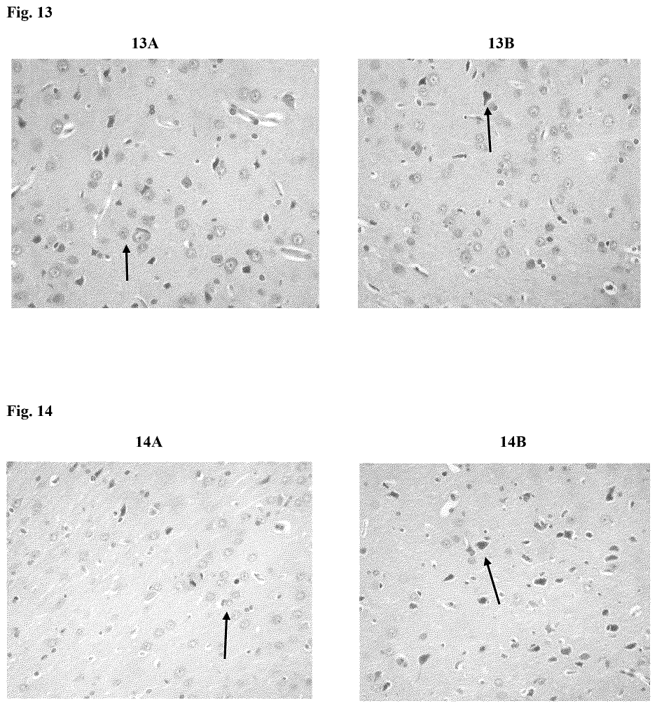

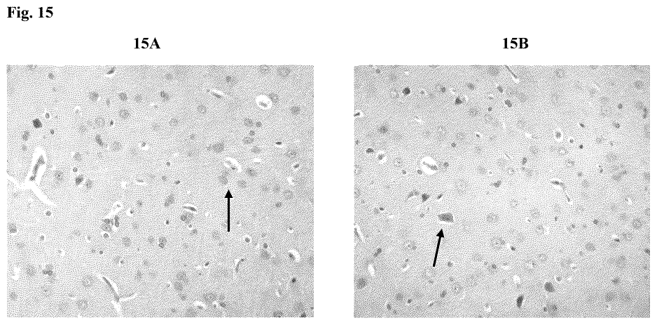

Administration of Gingerenone A, significantly improved brain pathology. Sections from substantia nigra for group with Gingerenone at 5 mg/kg (FIG. 12) showed both normal [FIG. 12A—Arrow] and degenerative neuronal cells having dark nucleus with mild basophilic cytoplasm [FIG. 12B—Arrow] and focal reactive microgliosis. Scant inflammation was noted at places. The neuropils had focal vacuoles. Sections from substantia nigra of animals administered with Gingerenone at 10 mg/kg (FIG. 13) showed both normal [FIG. 13A—Arrow] and degenerative neuronal cells having variable darkly stained nucleus [FIG. 13B—Arrow] with mild basophilic cytoplasm and scant inflammation. Sections from substantia nigra of animals administered with Gingerenone at 20 mg/kg (FIG. 14) showed both normal [FIG. 14A—Arrow] and degenerative neuronal cells having variable darkly stained nucleus [14B—Arrow] with mild basophilic cytoplasm and scant inflammation. In the Carbidopa controls (FIG. 15), sections from substantia nigra shows both normal [FIG. 15A—Arrow] and degenerative neuronal cells having dark nucleus with mild basophilic cytoplasm [FIG. 15B—Arrow]. Mild inflammation noted at places and the neuropils had focal vacuoles.

Overall administration of Gingerenone A resulted in improvement in locomotor and exploratory behaviors, improved working short-term memory and improved muscle rigidity as evidenced from the behavioural tests. Gingerenone A also improved the water homeostasis in brain by inhibiting AQP9 and increasing dopamine levels thereby alleviating the Parkinson's disease pathology suggesting that Gingerenone A is very effective for the management of Parkinson's disease.

### Example 3: Formulations

### Tables 15-24 Provide Illustrative Examples of Nutraceutical Formulations

TABLE 15
Tablet
Active Ingredients
Gingerenone A
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 16
Tablet
Active Ingredients
Gingerenone A
Hirsutenone
Excipients
Microcrystalline cellulose

TABLE 17
Capsule
Active Ingredients
Gingerenone A
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 18
Capsule
Active Ingredients
Gingerenone A, Hirsutenone
Excipients
Microcrystalline cellulose, Colloidal silicon dioxide, Magnesium stearate,
Polyvinylpyrrolidone/starch/Hydroxy propyl methyl cellulose, Hydroxy
propyl cellulose

TABLE 19
Powder
Active Ingredients
Gingerenone A/Hirsutenone
Excipients
BioPerine ®.

TABLE 20
Powder
Active Ingredients
Gingerenone A and Hirsutenone
Excipients
BioPerine ®,

TABLE 21
Gummy Formulation
Active Ingredients
Gingerenone A
Excipients
BioPerine ®, Gelatin (270 Bloom Mesh 10), Refined Sugar, Glucose Corn
Syrup, Citric Acid, Lactic Acid, Water, Natural Mango Flavor M38630,
Tartaric Acid, Refined Sugar

TABLE 22
Gummy Formulation
Active Ingredients
Gingerenone A
Hirsutenone
Excipients
Gelatin (270 Bloom Mesh 10), Refined Sugar, Glucose Corn Syrup, Citric
Acid, Lactic Acid, Water, Natural Mango Flavor M38630, Tartaric Acid,
Refined Sugar

TABLE 23
Candy Formulation
Active Ingredients
Gingerenone A
Excipients
BioPerine ®, Sucrose, Liquid Glucose, Flavoring agent, Menthol,
Acidulants (Citric acid/Tartaric Acid/Maleic Acid), Purified water

TABLE 24
Candy Formulation
Active Ingredients
Gingerenone A
Hirsutenone
Excipients
Sucrose, Liquid Glucose, Flavoring agent, Menthol, Acidulants (Citric
acid/Tartaric Acid/Maleic Acid), Purified water

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A method for maintaining or improving water homeostasis in the brain of a subject with a neurodegenerative condition, comprising a step of administering an effective dose of a composition comprising compounds of Formula I to the said subject, to bring about improvement in water homeostasis.

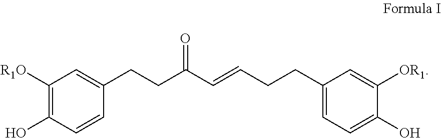

2. The method as in claim 1, wherein the compound is Gingerenone A, when R₁ is methyl in Formula I.

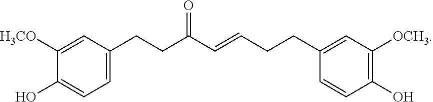 [SMILES: COc1cc(CC/C=C/C(=O)CCc2ccc(O)c(OC)c2)ccc1O]

3. The method as in claim 1, wherein the improvement of water homeostasis is brought about by modulating the expression of aquaporins (AQPs).

4. The method as in claim 3, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof.

5. The method as in claim 3, wherein the aquaporins are selected from the group consisting of AQP4 and AQP9.

6. The method as in claim 1, wherein improving water homeostasis results in improved cognitive and behavior patterns, improvement in neuronal plasticity and learning, reduction in inflammatory markers, improvement in neurotransmitter expression.

7. The method as in claim 1, wherein the neurodegenerative condition is selected from the group consisting of Alzheimer's disease, Parkinson's disease, Vascular dementia, neuromyelitis optica, Amyotrophic lateral sclerosis, prion diseases, idiopathic-normal pressure hydrocephalus, Lewy body dementia, and fronto-temporal dementia.

8. The method as in claim 1, wherein the subject is a mammal.

9. The method as in claim 1, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

10. A method for therapeutic management of Alzheimer's disease in a subject, comprising a step of administering an effective dose of a composition comprising compounds of Formula I to a subject with Alzheimer's disease.

11. The method as in claim 10, wherein the compound is Gingerenone A, when R₁ is methyl in Formula I.

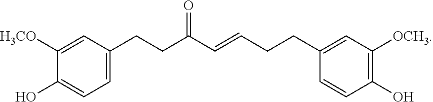 [SMILES: COc1cc(CC/C=C/C(=O)CCc2ccc(O)c(OC)c2)ccc1O]

12. The method as in claim 10, wherein management of Alzheimer's disease is brought about by improving water homeostasis by increasing the expression of aquaporins, improving cognition and behavioural patterns, decreasing plaque formation by reducing β-amyloid content, decreasing neurofibrillary tangles by reducing phosphorylated tau formation, decreasing apolipoprotein E levels, and reducing inflammatory markers.

13. The method as in claim 12, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof.

14. The method as in claim 12, wherein the aquaporins are selected from the group consisting of AQP4 and AQP9.

15. The method as in claim 10, wherein the subject is a mammal.

16. The method as in claim 10, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

17. A method for the therapeutic management of Parkinson's disease in a subject, comprising a step of administering an effective dose of a composition comprising compounds of Formula I to a subject with Parkinson's disease.

18. The method as in claim 17, wherein the compound is Gingerenone A, when R₁ is methyl in Formula I.

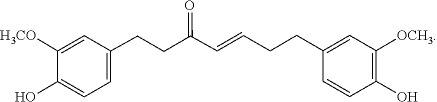 [SMILES: COc1cc(CC/C=C/C(=O)CCc2ccc(O)c(OC)c2)ccc1O]

19. The method as in claim 17, wherein management of Parkinson's disease is brought about by improving water homeostasis by increasing the expression of aquaporins, increasing dopamine levels, and reducing inflammatory markers.

20. The method as in claim 19, wherein the aquaporins are selected from the group consisting of AQP0, AQP1, AQP2, AQP3, AQP4, AQP5, AQP6, AQP8, AQP7, AQP9, AQP10, AQP11, AQP12, and combinations thereof.

21. The method as in claim 19, wherein the aquaporins are selected from the group consisting of AQP4 and AQP9.

22. The method as in claim 17, wherein the subject is a mammal.

23. The method as in claim 17, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.
